(19) United States
(12) Patent Application Publication
(10) Pub. No.: US 2014/0193352 A1
(43) Pub. Date: Jul. 10, 2014

(54) Packaged Product Including A Non-Aerosol Container And A Shaving Aid Composition Stored Within

(71) Applicant: EVEREADY BATTERY COMPANY INC., ST LOUIS, MO (US)

(72) Inventors: Pauley Chang, Milford, CT (US); Raymond Guimont, Guilford, CT (US)

(73) Assignee: EVEREADY BATTERY COMPANY INC., ST LOUIS, MO (US)

(21) Appl. No.: 14/150,097

(22) Filed: Jan. 8, 2014

Related U.S. Application Data
(60) Provisional application No. 61/750,530, filed on Jan. 9, 2013.
(60) Provisional application No. 61/858,173, filed on Jul. 25, 2013.

(51) Int. Cl. B65D 77/22; A61Q 9/02; A61K 8/04

(52) U.S. Cl. CPC B65D 77/22; A61K 8/042; A61Q 9/02
USPC 424/73; 53/467

(57) ABSTRACT

A packaged product and methods for manufacturing the same are provided. The packaged product includes a non-aerosol container and a shaving aid composition. The container includes a pocket. The shaving aid composition is in the form of a post-foaming gel and is stored within the pocket. The container is impermeable to the shaving aid composition during a useful life of the container. The shaving aid composition has a temperature dependent characteristic that enables the shaving aid composition to be stored within the pocket at temperatures below 48° C. without causing a volume of the shaving aid composition to significantly increase during the useful life of the container. The shaving aid composition can be stored within the pocket at temperatures below 48° C. without causing the volume of the shaving aid composition to increase to a magnitude such that the pocket is inadvertently opened during the useful life of the container.

### CROSS REFERENCE TO RELATED APPLICATIONS

This application claims the benefit of U.S. provisional patent applications Ser. Nos. 61/750,530, filed Jan. 9, 2013 and 61/858,173, filed Jul. 25, 2013, the contents of both being incorporated herein in their entireties for reference.

### BACKGROUND INFORMATION

1. Technical Field

Aspects of the present invention generally relate to packaged products, and more particularly relate to packaged products that include a non-aerosol container and a shaving aid composition stored therein.

2. Background Information

Shaving aid compositions have been provided in many forms, including creams, soaps, oils, foams, gels, and post-foaming gels. Post-foaming gels have been especially popular among users. Post-foaming gels are unique relative to other forms of shaving aid compositions in that they typically dispense from a container as a gel, and subsequently turn to a foam, for example after a user agitates the gel by rubbing the gel between his or her hands or by applying the gel to a skin surface. Historically, post-foaming gels typically have been stored within aerosol containers. Aerosol containers may be problematic in that they have a relatively high cost of goods. It would be desirable to provide a post-foaming gel in a non-aerosol container. Aspects of the present invention are directed to this and other problems.

### SUMMARY

According to an aspect of the present invention, a packaged product is provided that includes a non-aerosol container and a shaving aid composition. The container includes a pocket. The shaving aid composition is stored within the pocket. The shaving aid composition is in the form of a post-foaming gel. The container is impermeable to the shaving aid composition during a useful life of the container. The shaving aid composition has a temperature dependent characteristic that enables the shaving aid composition to be stored within the pocket at temperatures below 48° C. without causing a volume of the shaving aid composition to significantly increase during the useful life of the container, and that enables the shaving aid composition to be stored within the pocket at temperatures below 48° C. without causing the volume of the shaving aid composition to increase to a magnitude that causes the pocket to be inadvertently opened during the useful life of the container.

According to another aspect of the present invention, a shaving aid composition is provided. The shaving aid composition is in the form of a post-foaming gel. The shaving aid composition has a temperature dependent characteristic that enables the shaving aid composition to be stored within a pocket of a non-aerosol container at temperatures below 48° C. without causing a volume of the shaving aid composition to significantly increase during a useful life of the container, and that enables the shaving aid composition to be stored within the pocket at temperatures below 48° C. without causing the volume of the shaving aid composition to increase to a magnitude that causes the pocket to be inadvertently opened during the useful life of the container.

According to another aspect of the present invention, a non-aerosol container is provided. The container includes a pocket for storing a shaving aid composition therein. The shaving aid composition is in the form of a post-foaming gel. The container is impermeable to the shaving aid composition during a useful life of the container.

According to another aspect of the present invention, a method for manufacturing a packaged product is provided. The method includes the steps of: (1) providing a non-aerosol container having a pocket; (2) configuring the container so that the pocket is in an open configuration; (3) introducing into the pocket an amount of a shaving aid composition; and (4) configuring the container so that the pocket is in a closed configuration. The container is impermeable to the shaving aid composition during a useful life of the container. The shaving aid composition has a temperature dependent characteristic that enables the shaving aid composition to be stored within the pocket at temperatures below 48° C. without causing a volume of the shaving aid composition to significantly increase during the useful life of the container, and that enables the shaving aid composition to be stored within the pocket at temperatures below 48° C. without causing the volume of the shaving aid composition to increase to a magnitude that causes the pocket to be inadvertently opened during the useful life of the container.

This and other features and advantages of the present invention will become apparent in light of the drawings and detailed description of the present invention provided below.

### BRIEF DESCRIPTION OF THE DRAWINGS

FIG. 1 illustrates a packaged product.

FIG. 2 illustrates a sectional view of the packaged product shown in FIG. 1.

FIG. 3 illustrates the functionality of the opening element illustrated in FIG. 1.

FIG. 4 illustrates the packaged product shown in FIG. 1 having an alternative opening element.

FIG. 5 illustrates the packaged product shown in FIG. 1 having an alternative opening element.

FIG. 6 illustrates the functionality of the opening element illustrated in FIG. 5.

FIG. 7 illustrates the packaged product embodiment shown in FIGS. 1-3 during manufacturing.

FIG. 8 illustrates another packaged product.

FIG. 9 illustrates the functionality of the opening element illustrated in FIG. 8.

FIG. 10 illustrates another packaged product.

FIG. 11 illustrates the functionality of the opening element illustrated in FIG. 10.

### DETAILED DESCRIPTION

The present disclosure describes embodiments of a packaged product. The packaged product includes a non-aerosol container that includes a pocket with an amount of a shaving aid composition stored therein. The term “non-aerosol container” is used herein to mean a container that does not include, and is not designed to include, an internal pressure source (e.g., a propellant) operable to dispense a product (e.g., a shaving aid composition) from the pocket of the container. The container is impermeable to the shaving aid composition. The term “impermeable” is used herein relative to the container to describe that the shaving aid composition, or one or more ingredients of the shaving aid composition, will not escape the container during a useful life of the container in an amount that will appreciably negatively affect the functionality of the shaving aid composition. The term “useful life” is used herein to mean a duration of time, which duration of time may vary depending upon the particular application, but which in most instances will be at least one thousand (1000) days. The container may be configured so that the pocket is “closed” or “open”. In a “closed” configuration, the pocket is surrounded on all sides by one or more components of the container, thereby preventing the shaving aid composition stored in the pocket from exiting the pocket. In an “open” configuration, the container includes an opening through which the shaving aid composition may enter or exit the pocket.

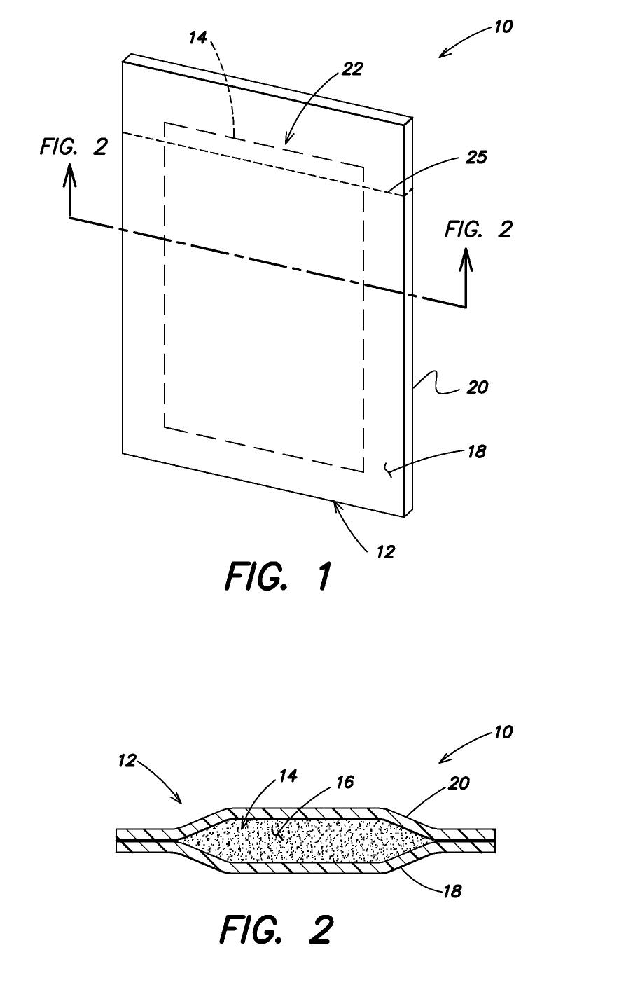

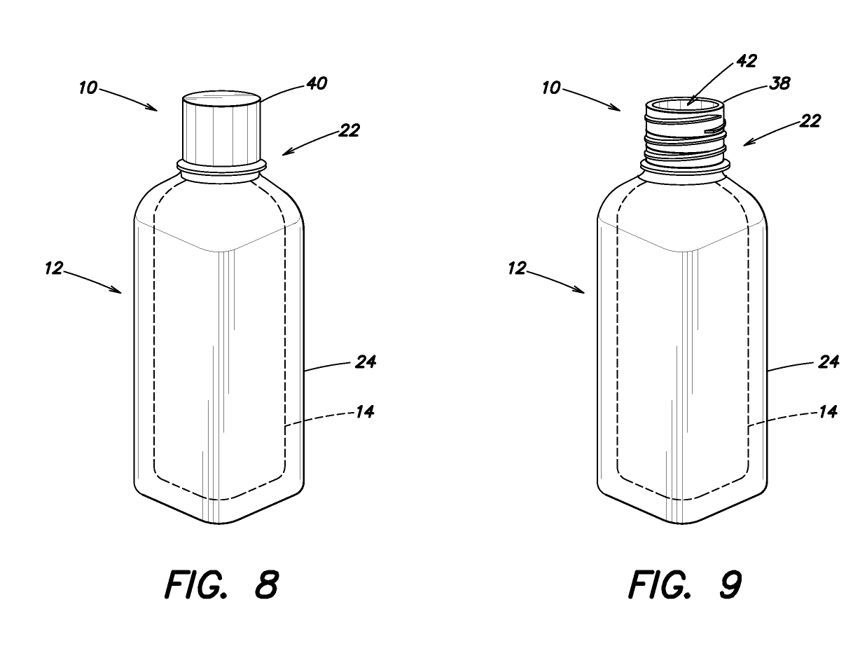

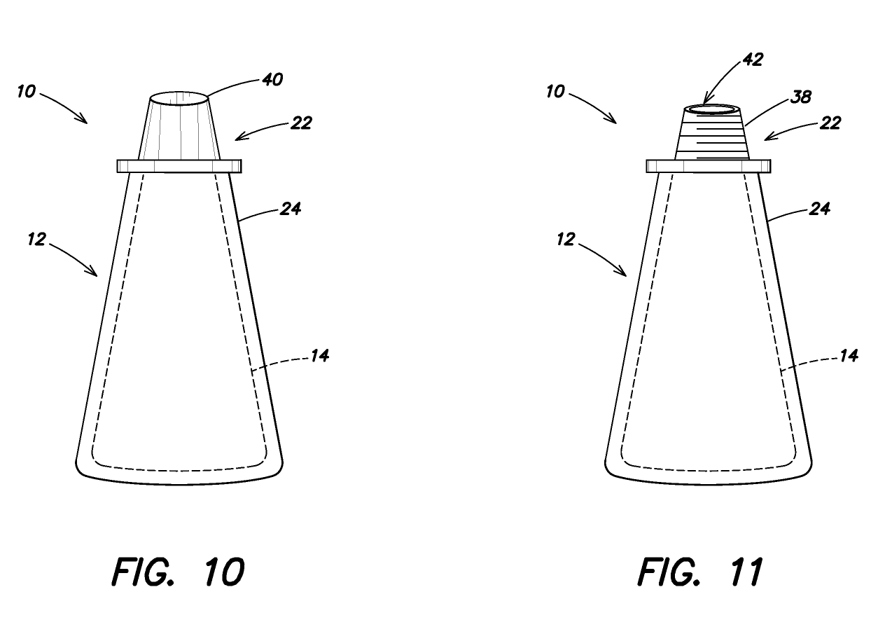

Now referring to embodiments shown in FIGS. 1-11, the packaged product 10 includes a non-aerosol container 12 that includes a pocket 14 with an amount of shaving aid composition 16 (see FIG. 2) stored therein. The pocket 14 is an internal cavity that is defined at least partially by one or more walls of the container 12. In FIGS. 1-7, for example, the container 12 is in the form of a sachet that includes a first wall 18 and a second wall 20 that are attached together to collectively define the pocket 14. The first and second walls 18, 20 may be attached to one another using any acceptable means or methods for attaching; e.g., the first and second walls 18, 20 may be attached to one another using adhesives, mechanical structures (e.g., fasteners), bonding techniques, welding techniques, or other means or methods. In some embodiments, the first and second walls 18, 20 may be integrally connected to one another along one or more edges of the container 12; e.g., the first and second walls 18, 20 may be a single component that is folded over, with the fold seam becoming an edge of the container 12. In some embodiments, the pocket 14 may be partially defined by an opening element 22 (see FIGS. 1, 3, 4, 5-6, and 8-11) that is included to aid a user in selectively opening the pocket 14. Examples of acceptable opening elements 22 are described in detail below. In some embodiments, the pocket 14 may be at least partially defined by one continuous wall of the container 12. In FIGS. 8 and 9, for example, the container 12 is in the form of a bottle that includes one continuous wall 24 and an opening element 22 that collectively define the pocket 14. In FIGS. 10 and 11, the container 12 is in the form of a tube that includes one continuous wall 24 and an opening element 22 that collectively define the pocket 14. In all embodiments, the container 12, or components thereof, are made from one or more materials that are impermeable to the shaving aid composition 16, thereby making the container 12 impermeable to the shaving aid composition 16. In FIGS. 1-7, for example, the first and second walls 18, 20 are made from a material that is impermeable to the shaving aid composition 16, thereby making the container 12 impermeable to the shaving aid composition 16. In other embodiments, the first and second walls 18, 20 may be permeable to the shaving aid composition 16, but a liner (not shown) that includes an impermeable material may be attached to the outer surfaces of the first and second walls 18, 20, thereby making the container 12 impermeable to the shaving aid composition 16. Acceptable materials that may be impermeable to the shaving aid composition 16 will be discussed in detail below.

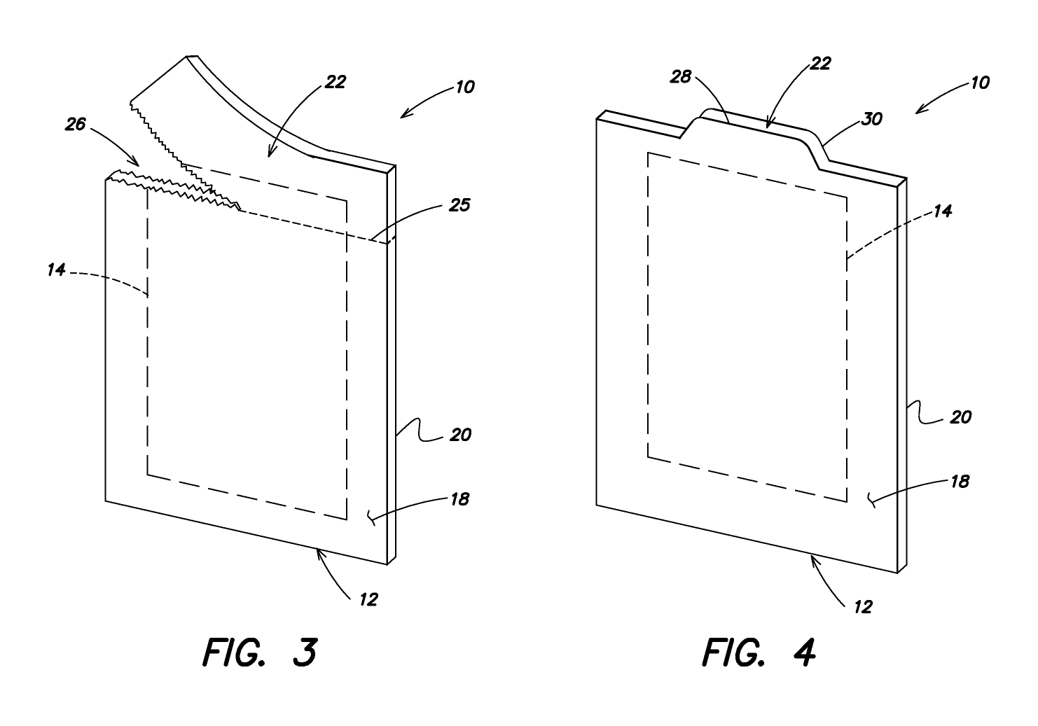

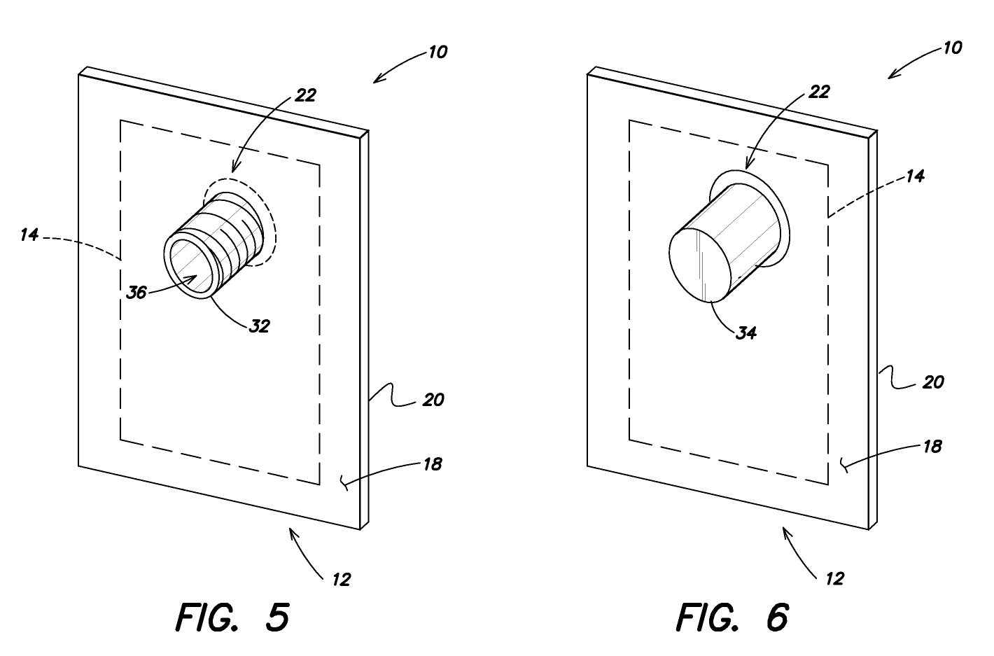

The container 12 is configured so that a user may selectively open the pocket 14 of the container 12. The pocket 14 can be selectively opened by a user in one or more different ways. Aspects of the present invention are not limited to any particular means or methods for selectively opening the pocket 14. Referring to FIG. 1, for example, the user may selectively open the pocket 14 by cutting one or both of the first and second walls 18, 20 with scissors or another acceptable device, or by tearing one or both of the first and second walls 18, 20 using his or her hands. As indicated above, the container 12 may include an opening element 22 that aids the user in selectively opening the pocket 14 of the container 12. Aspects of the present invention are not limited to use with any particular opening element 22. In the packaged product 10 illustrated in FIGS. 1-3, the opening element 22 includes a weakened tear line 25 disposed in the first and second walls 18, 20 of the container 12. During use of the packaged product 10 illustrated in FIGS. 1-3, the user may selectively open the pocket 14 by tearing the first and second walls 18, 20 along the weakened tear line 25. FIG. 3 illustrates the packaged product 10 with the first and second walls 18, 20 torn along a portion of the weakened tear line 25, thereby creating an opening 26 through which the shaving aid composition 16 stored in the pocket 14 may exit the pocket 14. The user may selectively retrieve (e.g., reach in and pick up) the shaving aid composition 16 from the pocket 14 through the opening 26, or the user may selectively dispense the shaving aid composition 16 from the pocket 14 through the opening 26. The user may selectively dispense the shaving aid composition 16 through the opening 26 to his or her hand, for example, by squeezing the first and second walls 18, 20 together. The user may then selectively apply the shaving aid composition 16 to the skin surface (e.g., the face, head, legs, etc.) that the user intends to shave. In FIG. 4, the opening element 22 includes a first tab portion 28 attached to and extending from the first wall 18, and a second tab portion 30 attached to and extending from the second wall 20. During use of the packaged product 10 shown in FIG. 4, the user may selectively open the pocket 14 of the container 12 by pulling the first and second tab portions 28, 30 apart from one another. This action causes the first and second walls 18, 20 to peel apart from one another and thereby creates an opening (not shown in FIG. 4) in the container 12 through which the shaving aid composition 16 stored in the pocket 14 may exit the pocket 14. In FIGS. 5 and 6, the opening element 22 includes a port 32 (see FIG. 5) attached to the first wall 18 of the container 12. A cap portion 34 (see FIG. 6) is removably attached to the port 32. During use of the packaged product 10 shown in FIGS. 5 and 6, the user may selectively open the pocket 14 of the container 12 by removing the cap portion 34 from the port 32. Removal of the cap portion 34 from the port 32 exposes an opening 36 (see FIG. 5) through which the shaving aid composition 16 stored in the pocket 14 may exit the pocket 14. In FIGS. 8-11, the opening element 22 includes a port 38 (see FIGS. 9 and 11) attached to the wall 24 of the container 12. A cap portion 40 (see FIGS. 8 and 10) is removably attached to the port 38. During use of the packaged products 10 shown in FIGS. 8-11, the user may selectively open the pocket 14 of the container 12 by removing the cap portion 40 from the port 38. Removal of the cap portion 40 from the port 38 exposes an opening 42 (see FIGS. 9 and 11) through which the shaving aid composition 16 stored in the pocket 14 may exit the pocket 14.

The container 12, or components thereof, are not limited to any particular shape or size. In FIGS. 1-7, the first and second walls 18, 20 are each generally planar and rectilinearly shaped. In alternative embodiments, the first and second walls 18, 20 may be at least partially non-planar and/or non-rectilinearly shaped. The pocket 14 is not limited to any particular shape or size; e.g., the pocket 14 can be shaped and sized to suit the application at hand. A useful pocket 14 volume for shaving aid applications may be between approximately two (2) milliliters (i.e., approximately seven one hundredths (0.07) of an ounce) to approximately one hundred eighteen (118) milliliters (i.e., approximately four (4) ounces) in magnitude. Although the drawings show the container 12 in the form of a sachet (see FIGS. 1-7), a bottle (see FIGS. 8 and 9), and a tube (see FIGS. 10 and 11), the container 12 need not be in any particular form.

The container 12, or components thereof, need not be made from any particular material(s) that is impermeable to the shaving aid composition 16. The particular material(s) used may depend on the application at hand. For example, the particular material(s) may depend on the ingredients of the shaving aid composition 16. The ingredients of the shaving aid composition 16 are discussed in detail below. The following materials are examples of acceptable materials that may be impermeable to the shaving aid composition 16: PD-86246M laminate, offered by Oracle Packaging of Winston-Salem, N.C., U.S.A; EAA3.0 laminate, offered by Glenroy, Inc. of Menomonee Falls, Wis., U.S.A; and polyethylene terephthalate (PET), which is a known thermoplastic polymer. Other acceptable materials, including polymers, metallised films, metallised laminates, etc., may be impermeable to the shaving aid composition 16. Further acceptable materials include metals, e.g. aluminum, which can additionally be provided with an impermeable liner. Aluminum has particular utility for the wall 24 of the container 12 of the tube depicted in FIGS. 10 and 11. The wall 24 is generally permanently deformable and will not resiliently recover after a portion of the shaving aid composition is dispensed from the pocket 14 such that a void is introduced into the pocket 14 whereinto the volume of the shaving aid composition might increase. The PD-86246M laminate and the EAA3.0 laminate described above are similar in that they both include an amount of aluminum. The inclusion of aluminum or another metal may at least partially enable a material to be impermeable to the shaving aid composition 16. The one or more components of the container 12 that are impermeable to the shaving aid composition 16 need not include aluminum or another metal, however.

The one or more materials used to make the container 12, or components thereof, may be selected so that the container 12, or components thereof, have one or more desired characteristics. For example, it may be desirable that the container 12, or components thereof, be suitable for printing and/or affixing a product label thereon. It may be desirable that the container 12, or components thereof, be easily tearable or breakable by the user. It may be desirable that the container 12, or components thereof, be non-slippery when wet. It may be desirable that the container 12, or components thereof, be resiliently deformable. It may be desirable that the container 12, or components thereof, be permanently deformable. Referring to FIGS. 1-7, for example, it may be desirable that the first and second walls 18, 20 be resiliently deformable so that the volume of the pocket 14 may increase or decrease as the volume of the shaving aid composition 16 stored within the pocket 14 increases or decreases, respectively.

The shaving aid composition 16 is in the form of a post-foaming gel. Being in the form of a post-foaming gel, the shaving aid composition 16 exits the pocket 14 of the container 12 as a gel, and subsequently turns to a foam, for example after a user agitates the shaving aid composition 16 by rubbing the shaving aid composition 16 between his or her hands. The shaving aid composition 16 is a mixture that includes at least a gelling agent and a post-foaming agent. The gelling agent is at least partially responsible for causing the shaving aid composition 16 to be in the form of a gel when it exits the pocket 14 of the container 12. Aspects of the present invention are not limited to use with any particular gelling agent. Examples of acceptable gelling agents will be described in detail below. The post-foaming agent included in the shaving aid composition 16 is at least partially responsible for causing the shaving aid composition 16 to turn from a gel to a foam, for example after a user agitates the shaving aid composition 16. The post-foaming agent will be described in detail below.

The shaving aid composition 16 has one or more temperature dependent characteristics that enable the shaving aid composition 16 to be stored within the pocket 14 of the container 12 during a useful life of the container 12. In particular, the temperature dependent characteristics enable the shaving aid composition 16 to be stored within the closed pocket 14 at temperatures below approximately forty-eight degrees Celsius (48° C.) (i.e., below approximately one hundred twenty degrees Fahrenheit (120° F.)) without causing a volume of the shaving aid composition 16, and thus a volume of the pocket 14, to significantly increase during the useful life of the container 12. The volume of the shaving aid composition 16 significantly increases if it increases from an initial volume to a subsequent volume that is greater than approximately thirty percent (30%) of the initial volume. The initial volume of the shaving aid composition 16 may be the volume of the shaving aid composition 16 when it is first introduced into the pocket 14 and the pocket 14 is closed during manufacture of the packaged product 10, as described below. The subsequent volume of the shaving aid composition 16 may be the volume of the shaving aid composition 16 at a later time during the useful life of the container 12. The temperature dependent characteristics of the shaving aid composition 16 also enable the shaving aid composition 16 to be stored within the closed pocket 14 at temperatures below approximately forty-eight degrees Celsius (48° C.) (i.e., below approximately one hundred twenty degrees Fahrenheit (120° F.)) without causing a volume of the shaving aid composition 16 to increase to a magnitude that causes the pocket 14 to be inadvertently opened during the useful life of the container 12. The pocket 14 may inadvertently open, for example, if a volume of the shaving aid composition 16, and thus a volume of the pocket 14, becomes so great in magnitude that the structural integrity of the container 12 is compromised, thereby causing the pocket 14 to open. An example of a temperature dependent characteristic of the shaving aid composition 16 is the vapor pressure of the shaving aid composition 16. The vapor pressure of the shaving aid composition 16 may be within the range of approximately negative forty-one thousand (−41,000) Pascals gauge and negative twenty-one thousand (−21,000) Pascals gauge at approximately twenty-one degrees Celsius (21° C.) (i.e., within the range of approximately negative six (−6) psi gauge and negative three (−3) psi gauge at approximately seventy degrees Fahrenheit (70° F.)); and/or the vapor pressure of the shaving aid composition 16 may be within the range of approximately fifty-five hundred (5,500) Pascals gauge and forty-one thousand (41,000) Pascals gauge at approximately thirty-seven degrees Celsius (37° C.) (i.e., within the range of approximately one (1) psi gauge and six (6) psi gauge at approximately one hundred degrees Fahrenheit (100° F.)). Another example of a temperature dependent characteristic of the shaving aid composition 16 is the coefficient of thermal expansion of the shaving aid composition 16.

The ingredients of the shaving aid composition 16, and/or the relative amount of each ingredient of the shaving aid composition 16, are selected so that the shaving aid composition 16 has one or more temperature dependent characteristics as described above. For example, whereas post-foaming gels typically include a post-foaming agent that includes normal butane and/or iso-butane (individually and collectively referred to hereinafter as “butane”), the present shaving aid composition 16 is substantially free of butane. The phrase “substantially free of butane” is used herein relative to the shaving aid composition 16 to describe that butane is not present in the shaving aid composition 16 in sufficient quantities that will appreciably negatively affect the one or more temperature dependent characteristics of the shaving aid composition 16. The shaving aid composition 16 may be substantially free of butane, for example, if the post-foaming agent of the shaving aid composition 16 includes less than seven tenths (0.7) percent weight by volume of butane. The post-foaming agent of the shaving aid composition 16 consists substantially of a pentane agent. The phrase “consists substantially of a pentane agent” is used herein relative to the post-foaming agent of the shaving aid composition 16 to describe that, to the extent the post-foaming agent includes materials other than the pentane agent, those materials are not present in the post-foaming agent in sufficient quantities that will appreciably negatively affect the one or more temperature dependent characteristics of the shaving aid composition 16. The pentane agent includes one or more of the three known pentane isomers: normal pentane, isopentane, neopentane. In one pentane agent embodiment, the pentane agent includes: ninety (90) moles of normal pentane for every ten (10) moles of neopentane. This pentane agent embodiment may enable the shaving aid composition 16 to have a vapor pressure of approximately negative thirty-four thousand (−34,000) Pascals gauge at approximately twenty one degrees Celsius (21° C.) (i.e., approximately negative four and nine tenths (−4.9) psi gauge at approximately seventy degrees Fahrenheit (70° F.)). In some embodiments, the relative amounts of the gelling agent and the post-foaming agent in the shaving aid composition 16 enables the shaving aid composition 16 to have one or more temperature dependent characteristics as described above. The shaving aid composition 16 may preferably include between ninety five (95) percent weight by volume and ninety nine (99) percent weight by volume of the gelling agent, and between one (1) percent weight by volume and five (5) percent weight by volume of the post-foaming agent.

A first example of the shaving aid composition 16 includes ninety-seven (97) percent weight by volume of a gelling agent having the following ingredients:

| | Percent weight by volume |
|---|---|
| Deionized water | 66.25 |
| Palmitic acid | 10.00 |
| Triethanolamine | 8.40 |
| Polyvinylpyrrolidone K-90 | 0.60 |
| Stearic acid | 3.50 |
| Sunflower oil monoglyceride | 5.00 |
| Sorbitol 70%, USP | 3.00 |
| Polyox ® WSR 301 | 0.25 |
| Aloe vera powder | 0.20 |
| Vitamin E acetate USP | 0.30 |
| Glycerine, USP | 2.00 |
| Fragrance | 0.50 |
| Total | 100.00 |

The first example of the shaving aid composition 16 also includes three (3) percent weight by volume of a post-foaming agent. That post-foaming agent consists entirely of a pentane agent having the following ingredients:

| | Percent weight by volume |
|---|---|
| Normal pentane | 87.50 |
| Neo-pentane | 12.50 |
| Total | 100.00 |

A second example of the shaving aid composition 16 includes ninety-five (95) percent weight by volume of a gelling agent having the following ingredients:

| | Percent weight by volume |
|---|---|
| Deionized water | 65.40 |
| Palmitic acid | 10.00 |
| Triethanolamine | 8.50 |
| Glycerine, USP | 3.00 |
| Stearic acid | 3.50 |
| Sunflower oil monoglyceride | 5.00 |
| Sorbitol 70%, USP | 3.00 |
| Colloidal Oatmeal | 0.30 |
| Hydroxyethyl Cellulose | 0.30 |
| DL-panthenol | 0.50 |
| Fragrance | 0.50 |
| Total | 100.00 |

The second example of the shaving aid composition 16 also includes five (5) percent weight by volume of a post-foaming agent. That post-foaming agent consists entirely of a pentane agent having the following ingredients:

| | Percent weight by volume |
|---|---|
| Iso-pentane | 75.00 |
| Normal pentane | 25.00 |
| Total | 100.00 |

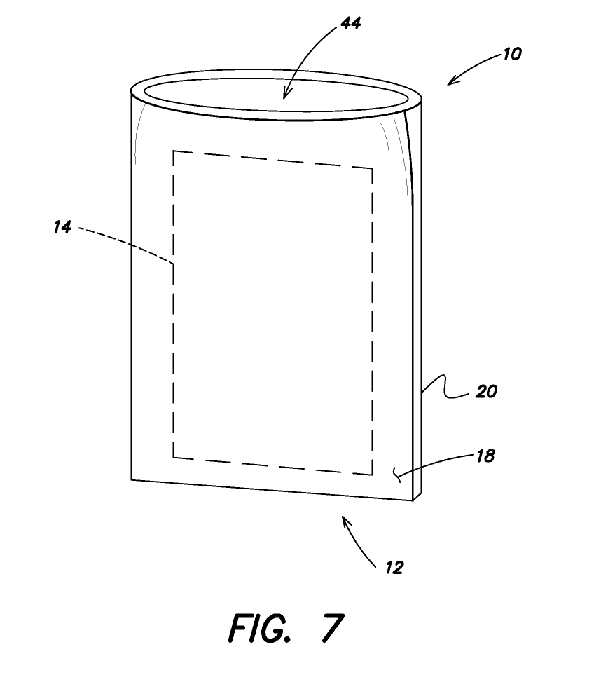

The packaged product 10 can be manufacturing in one or more different ways. One acceptable method for manufacturing the packaged product 10 includes the steps of: (1) configuring the container 12 so that the pocket 14 is open; (2) introducing the shaving aid composition into the pocket 14 for storage; and (3) configuring the container 12 so that the pocket 14 is closed. In manufacturing the packaged product 10 illustrated in FIGS. 1-3, the container 12 may be provided as shown in FIG. 7, with the first and second walls 18, 20 unattached along one edge of the container 12. In FIG. 7, an opening 44 is formed through which the shaving aid composition 16 may be introduced into the pocket 14. After the shaving aid composition 16 is introduced into the pocket 14, the first and second walls 18, 20 may be attached along all edges so that the pocket 14 is closed. The pocket 14 may then remain closed until a user selectively opens the pocket 14 as described above.

While various embodiments of the present invention have been disclosed, it will be apparent to those of ordinary skill in the art that many more embodiments and implementations are possible within the scope of the invention. For instance, features disclosed in connection with any one embodiment can be used alone or in combination with each feature of the respective other embodiments. Accordingly, the present invention is not to be restricted except in light of the attached claims and their equivalents.

What is claimed is:

1. A packaged product, comprising:
a non-aerosol container that includes a pocket; and
a shaving aid composition stored within the pocket;
wherein the shaving aid composition is in the form of a post-foaming gel;
wherein the container is impermeable to the shaving aid composition during a useful life of the container; and
wherein the shaving aid composition has a temperature dependent characteristic that enables the shaving aid composition to be stored within the pocket at temperatures below 48° C. without causing a volume of the shaving aid composition to significantly increase during the useful life of the container, and that enables the shaving aid composition to be stored within the pocket at temperatures below 48° C. without causing the volume of the shaving aid composition to increase to a magnitude that causes the pocket to be inadvertently opened during the useful life of the container.

2. The packaged product of claim 1, wherein the shaving aid composition is substantially free of butane.

3. The packaged product of claim 1, wherein the shaving aid composition includes a post-foaming agent that consists substantially of a pentane agent.

4. The packaged product of claim 3, wherein the pentane agent includes a compound selected from the group consisting of: normal pentane, isopentane, neopentane.

5. The packaged product of claim 1, wherein the temperature dependent characteristic of the shaving aid composition is a vapor pressure, wherein the vapor pressure of the shaving aid composition is within the range of −41,000 Pascals gauge and −21,000 Pascals gauge at 21° C.

6. The packaged product of claim 1, wherein the characteristic of the shaving aid composition is a vapor pressure, wherein the vapor pressure of the shaving aid composition is within the range of 5,500 Pascals gauge and 41,000 Pascals gauge at 37° C.

7. The packaged product of claim 1, wherein the characteristic of the shaving aid composition is an expansion coefficient.

8. The packaged product of claim 1, wherein the container includes material that is impermeable to the shaving aid composition, and wherein the material includes an amount of aluminum.

9. The packaged product of claim 1, wherein the container is configured so that a user may selectively open the pocket.

10. The packaged product of claim 9, wherein the container includes an opening element that aids the user in selectively opening the pocket.

11. The packaged product of claim 1, wherein the volume of the pocket is within the range of 2 milliliters and 118 milliliters.

12. The packaged product of claim 1, wherein the volume of the shaving aid composition significantly increases if it expands from an initial volume to a subsequent volume that is greater than 30 percent of the initial volume.

13. The packaged product of claim 1, wherein the container includes a wall that at least partially defines the pocket.

14. The packaged product of claim 1, wherein the useful life of the container is a time period having a duration greater than 1000 days.

15. A shaving aid composition in the form of a post-foaming gel, the shaving aid composition having a temperature dependent characteristic that enables the shaving aid composition to be stored within a pocket of a non-aerosol container at temperatures below 48° C. without causing a volume of the shaving aid composition to significantly increase during a useful life of the container, and that enables the shaving aid composition to be stored within the pocket at temperatures below 48° C. without causing the volume of the shaving aid composition to increase to a magnitude that causes the pocket to be inadvertently opened during the useful life of the container.

16. A non-aerosol container, the container comprising:
a pocket for storing a shaving aid composition therein;
wherein the shaving aid composition is in the form of a post-foaming gel;
wherein the container is impermeable to the shaving aid composition during a useful life of the container.

17. A method for manufacturing a packaged product, comprising the steps of:
providing a non-aerosol container having a pocket;
configuring the container so that the pocket is in an open configuration;
introducing into the pocket an amount of a shaving aid composition; and
configuring the container so that the pocket is in a closed configuration;
wherein the container is impermeable to the shaving aid composition during a useful life of the container; and
wherein the shaving aid composition has a temperature dependent characteristic that enables the shaving aid composition to be stored within the pocket at temperatures below 48° C. without causing a volume of the pocket to significantly increase during the useful life of the container, and that enables the shaving aid composition to be stored within the pocket at temperatures below 48° C. without causing the volume of the shaving aid composition to increase to a magnitude that causes the pocket to be inadvertently opened during the useful life of the container.
